(19) United States
(12) Patent Application Publication
(10) Pub. No.: US 2012/0245410 A1
(43) Pub. Date: Sep. 27, 2012

(54) LOTUS MASSAGE SYSTEMS AND METHODS

(76) Inventor: Sean Davis, Wilmington, DE (US)

(21) Appl. No.: 13/491,089

(22) Filed: Jun. 7, 2012

Related U.S. Application Data
(63) Continuation-in-part of application No. 12/565,086, filed on Sep. 23, 2009.
(60) Provisional application No. 61/107,461, filed on Oct. 22, 2008.

(51) Int. Cl. A61M 21/00; A61H 7/00

(52) U.S. Cl. 600/28; 601/136

(57) ABSTRACT

The Lotus massage systems and methods disclosed herein include various combined components that enhance the overall massage experience. These components integrate choreographed, mapped, synchronized, and freeform application of massage techniques with music, silence, ambient noise reduction, 360 degree site cancellation, aroma therapy, etc. The massage systems and methods disclosed herein may also involve choreographed, mapped, synchronized and/or freeform application of strokes and movements, by referencing one or more music body charts.

### RELATED APPLICATIONS

This application is a continuation-in-part of Ser. No. 12/565,086, filed on Sep. 23, 2009, which claims priority to provisional application Ser. No. 61/107,461 filed on Oct. 22, 2008. Each of the aforementioned applications is incorporated herein by reference in its entirety.

### BACKGROUND

1. Field of the Invention

The present invention relates to massage systems and methods, and particularly massage systems and methods utilizing choreographed, mapped, synchronized and/or freeform application of strokes, movements, and stance in sequences and in combination with music.

2. Background

Massage is well-known for the physiological and psychological benefits it provides. It is used by many as a proven way to reduce stress, alleviate pain, improve circulation, release endorphins, and relax the body and mind. Massage received consistently over significant periods has even been shown to lower blood pressure, reduce recovery time after injury, improve sleeping, and increase concentration. Some even claim that massage provides emotional and spiritual balance, by producing a meditative state and a heightened awareness of living in the present.

Music is also known for providing physiological and psychological benefits similar to those provided by massage. Health practitioners, in some cases, prescribe musical therapy to reduce pain and symptoms associated with cancer treatments, for example. Related studies have shown that those undergoing music therapy also experience reductions in high blood pressure, depression, and insomnia. The physiological effects of music were first observed in the 17th Century, when a French musician noted changes to his pulse as he sang in different tempos. More recent studies have discovered that faster tempos elicit a greater physiological response than slower paced music. Music is also known for its ability to stimulate endorphin release.

Although certain types of sounds may ease anxiety and reduce stress for some individuals, for many music still remains a personal preference. Some believe that music preferences play a significant part in the physiological effectiveness of music on the listener. Scientists have shown that harmonic relations and tones are determined by the right brain auditory cortex and organization of sound is controlled by the left brain auditory cortex. Diverse cultures, however, recognize different melodies and harmonies, and thus have different physiological and psychological responses to music. Musical selections, which are preferred to be heard by a listener, result in increased pleasure and improved benefits because they are more easily recognized and organized by the listener's brain.

Because of the numerous physiological and psychological benefits associated with both massage and music, massage practitioners frequently play music during massage sessions. A massage recipient receiving a massage, however, usually does not have any input into the type of music played during the session. Rather, musical selections are predetermined without acknowledgement of the massage recipient's personal preferences or the potential physiological and psychological influence on the recipient. Massage practitioners typically only consider the specific massage modality they are using—without considering any type of mapping or synchronization of their strokes and movements with music played during the session. Although various types of massage modalities provide benefits, there is a clear need for improved massage systems and methods that seek to combine massage and music for an enhanced massage experience. The present invention fulfills this need and provides further related advantages, as described below.

### SUMMARY

The Lotus massage systems and methods disclosed herein include various combined components that enhance the overall massage experience. These components integrate choreographed, mapped, synchronized, and freeform application of massage techniques with music, silence, ambient noise reduction, 360 degree site cancellation, aromatherapy, etc. The massage systems and methods disclosed herein may also involve choreographed, mapped, synchronized and/or freeform application of strokes and movements, by referencing one or more music body charts. Music body charts can provide massage practitioners with a tool for matching audible, note/tonal frequency and resonance changes in timing, notes and tones, scale progression, breakdowns, sustained notes, chords and sporadic sounds with massage techniques such as stroke values, pressure, direction and speed.

A more complete understanding of various embodiments of Lotus massage systems and methods will be afforded to those skilled in the art, as well as a realization of additional advantages and objects thereof, by consideration of the following detailed description. Reference will be made to the appended sheets which will first be described briefly.

### BRIEF DESCRIPTION OF THE DRAWINGS

The drawings described herein are for illustrative purposes only and are not intended to limit the scope of the present disclosure. In the drawings:

FIG. 1a shows a massage recipient and a massage practitioner utilizing an auditory system during a massage session;

FIG. 1b shows another view a massage recipient and a massage practitioner utilizing an auditory system during a massage session;

FIG. 2 shows how an auditory system, having left and right speakers, corresponds with a mid-sagittal plane;

FIG. 3 shows one version of a music body chart for an upper torso;

FIG. 4 shows another version of a music body chart for an upper torso;

FIG. 5 show one version of a music body chart for an upper extremity;

FIG. 6 shows one version of a music body chart for a hand;

FIG. 7 shows one version of a music body chart for a lower extremity;

FIG. 8 shows another version of a music body chart for a lower extremity; and

FIG. 9 shows one version of a music body chart for a foot.

### DETAILED DESCRIPTION

Lotus massage systems and methods include multi-faceted massage techniques which may be adapted to all areas of the massage. These areas can therefore extend to the spa industry and health care industry, including application during physical therapy, esthetician massage, chiropractic care, pre- and post-operative therapy, pre- and post chemotherapy, autism therapy, pediatric care, etc. The foundation of Lotus systems and methods combines benefits associated with both music and massage to heighten and intensify the massage experience.

As used herein the term “Lotus” is defined as combining music and applying pressure, such as those used in various types of massage modalities, through mapping, choreography, synchronization, and freeform application. The term “Lotus” may also apply to the use of music body charts, configured for use as a practitioner's tool. This tool allows practitioners to correspond audible, note/tonal frequency and resonance changes in timing, notes and tones, scale progression, breakdowns, sustained notes, chords and sporadic sounds with massage techniques, stroke values, pressure, direction, and speed.

As used herein, the term “music” is to be broadly defined as any vibration or audible sound produced by any source in succession, combination, or one or more temporal relationships to produce rhythm, melody, harmony, and other sonic qualities. Other elements of music encompassed by this definition include pitch, tempo, meter, articulation, dynamics, timbre and texture.

The term “massage” is broadly defined herein as the application of pressure to any body surface. The term, therefore, includes the application of various types of massage modalities, including, but not limited to Swedish massage, Acupressure, Trigger Point Therapy, Deep Tissue massage and Amma. The massage techniques described herein also include variations and combinations of the aforementioned modalities. Moreover, in using the systems and methods disclosed herein massage practitioners may incorporate their own massage techniques and styles without limitation. Commercially, Lotus massage systems and methods described herein may be collectively known as the LOTUS SYSTEM®.

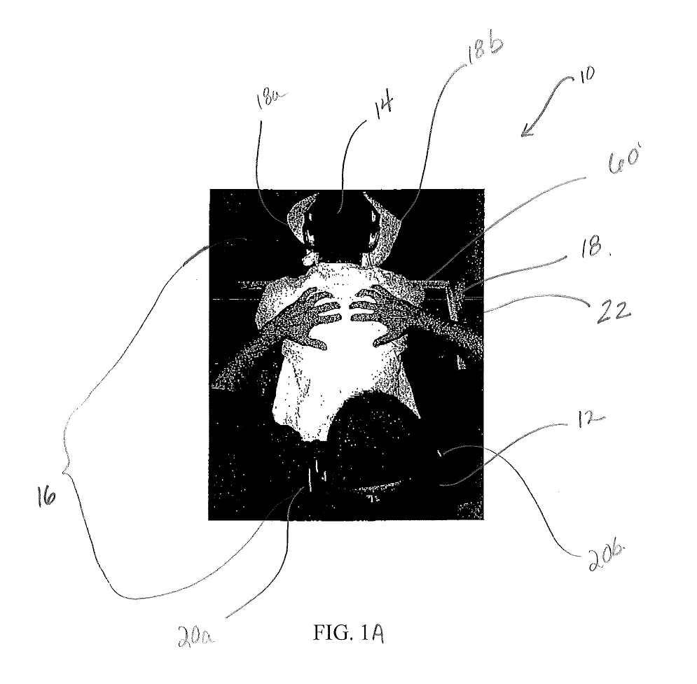

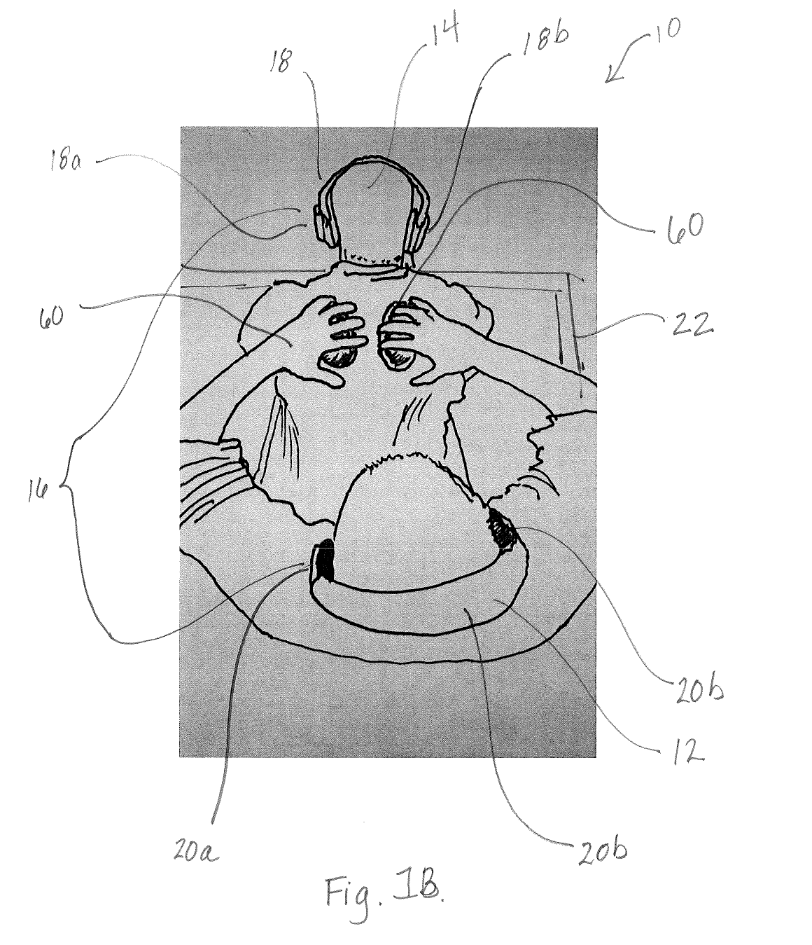

Turning in detail to the drawings, FIGS. 1A-9 show various components of Lotus massage systems and methods. FIGS. 1A and 1B show implementations of the Lotus massage system 10 where a massage practitioner 12 and a massage recipient 14 use an auditory system 16. In this version, the auditory system 16 includes two pairs of auditory devices such as headphones. Thus, there are at least two components of the auditory system: (1) a massage recipient headphone pair 18, having a left speaker 18a and a right speaker 18b and (2) a massage practitioner headphone pair 20, having a left speaker 20a and a right speaker 20b. Although the headphones are shown as covering the ears, the shape and style of headphones used are not to be construed as limiting. The headphones worn by either the massage recipient or practitioner may be any shape and configuration. In addition, headphones may not necessarily be coupled to a practitioner's or recipient's head and may include, for example, earbuds which are partially or fully disposed within at least one of the wearer's ear canal. At least one speaker, however, is configured to output a musical selection.

In addition, the auditory devices used are preferably wireless such that the comfort and positioning of the recipient and/or the practitioner during the massage session is optimized. In another version of the system, the massage recipient may be positioned on a supportive device 22 such as a table or chair used for massage. The supportive device 22 may be configured to output a musical selection such that the recipient can hear the musical selection and/or feel vibrations resulting from output of the musical selection.

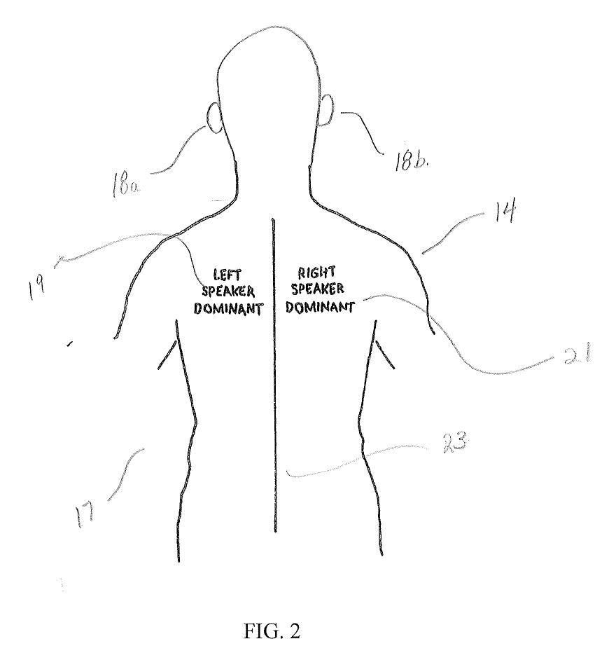

FIG. 2 shows a massage recipient's upper torso 24 and the preferred relationship of how the recipient's left speaker 18a and right speaker 18b can correspond to the massage recipient's upper torso 17. Here the upper torso has a left speaker dominant side 19 and a right speaker dominant side 21, where the upper torso is separated by a mid-sagittal plane 23. Knowing the arrangement of the musical selection can, therefore, be of particular importance during practice of Lotus massage systems and methods, as further described below.

In one version of the LOTUS SYSTEM®, the auditory system 16 is configured to output music into headphones or similar devices, having left and right speakers 18a, 18b. Use of speakers of this type is known to provide the massage recipient with a heightened sense of transference. A heightened sense of transference is used herein to mean a state or sense that occurs when there is a substantial removal of sounds surrounding the massage recipient, for example, sounds from the recipient's audible ambient environment which may distract the recipient. In contrast, under “normal” listening circumstances, such as when a recipient listens to music without the use of headphones, it is often difficult for the listener to fully engage with the music, and perhaps be distracted from the musician's true intentions. Under normal listening circumstances there is often too much audible, ambient noise in between the external speaker and the listener's ear. With the use of headphones, a recipient can become removed from the audible ambient environment and allowed to capture all of the composition's inherent qualities without outside distractions. As such, transference of vibrations and musical notes can cause the massage recipient's mind and body to briefly separate into right and left sides, and/or blend back and forth through the speakers providing a feeling of movement within the body. For example, when music emanates into one specific side of a speaker, the massage practitioner may massage on that side. Whether a sustained note quickly transitions from one side to the other, the massage practitioner can move with the music to further enhance the feeling of movement within the body. To ensure the recipient experiences transference, a massage practitioner should be familiar with the arrangement of the musical selection and when notes from the selection are played from a specific speaker.

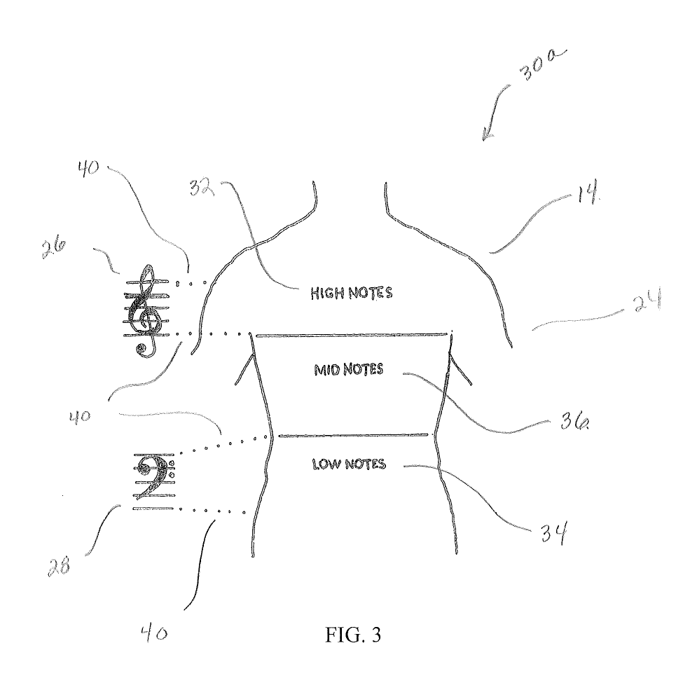

When a musical selection is played, a note is produced for a specified duration, creating a vibrational frequency and resonance known as “measure.” Time signatures within one or more measures are known as “meters” in which a regular pattern reoccurs throughout music selection. The regular pattern is referred to herein as “time.” Time signatures are separated into a treble clef 26 and a bass clef 28, which are represented in FIGS. 3-9. Moreover, treble clef notes, as used herein, are those notes above middle C vibration and frequency and bass clef notes are those notes below middle C vibration and frequency. Clef type and notes, however, change depending on the nature and genre of the musical selection.

In some persons, vibrational frequencies and resonances of notes and tones can produce various emotional reactions as well as fluctuations in blood pressure. Considering these potential reactions, it is believed that use of Lotus massage systems and methods has the ability to shift emotional states by combining massage techniques with musical changes in notes, tones, rhythm and tempo patterns, chords, scale progression, bridges, breakdowns, sustained notes, chords and sporadic sounds heard by the massage recipient. For example, vibrational frequencies and resonances of musical selections may cause feelings of happiness, sadness, contemplation, rage and/or anger. The following provides a broad range of how aspects of a musical section can affect a person's emotional state:
happiness=fast, staccato
sadness=slow, legato
anger=fast, legato
fear=slow, staccato.

As used herein the term “staccato” indicates notes produced in a detached and distinctly separate manner, where silence consists of a latter part of the time allocated for each produced note. Rhythm is not typically affected when staccato notes are produced. Notes identified as staccato are usually abrupt and short. In musical compositions, staccato notes are notated by a dot over the head of the note when the stem is downward, or by a dot below the head of the note when the stem is upward. Examples of musical selections that use staccato notes include: Communication Breakdown by Led Zeppelin, Anemia by Tool, and Indestructible by Disturbed. Other genres with musical selections having staccato-type notes include Rock, Hard Rock, Reggae, Jazz, Rhythm and Blues (R&B), Dixieland, and Classical.

As used herein the term “legato” indicates notes produced without breaks, i.e. in a smooth and connected manner. Some genres having musical selections with legato-type influences include Soft Rock, Ballads, Smooth Jazz, Heavy Metal, and Classical. Other genres, such as Rock for example, frequently use staccato and legato notes in one musical selection. Musical artists that have release musical selections combining staccato and legato notes include Black Sabbath, Jethro Tull, Yes, Pink Floyd, Rush, the Allman Brothers Band, and Jimi Hendrix.

Certain musical selections are known for their effect on triggering memories and emotional, autonomic and limbic responses. Known selections of this type include: the Electric Slide, the Macarena, the Chicken Dance, Everybody Dance Now, Wind Beneath My Wings, and Daddy's Little Girl. Other musical selections are known for their ability to generate a para-sympathetic response which can cause a listener to have “goose bumps.” These musical selections include: The Marine Corps Hymn, Taps, God Bless America, various National Anthems, and various musical selections by Mozart.

Through output of musical selections such as those referred to above in combination with choreographed, mapped, synchronized, and/or freeform application of massage techniques a massage recipient can have an enhanced massage experience. As such, Lotus massage systems and methods can include any and all strokes, movements, and stances practiced by massage practitioners during a massage session. The following list provides some type strokes by massage practitioners during massage and their corresponding meaning:
Effleurage (gliding strokes)
Petrissage (kneading and compression strokes)
Friction (deep circular rubbing)
Tapotement (percussion tapping, pounding/used in the massage modality Amma)
Vibration (very fine, rapid shaking)
Acupressure (broader focused pressure in one area)
Trigger Point (localized, focused pressure on a “knot”)

FIGS. 3-9 and show various embodiment of one component of Lotus massage systems and methods—a music body chart 30. Massage practitioners can apply the stroke types listed above, for example, by mapping, choreography, and/or freeform application, by referencing a music body chart, while listening to a musical selection. Music body charts used in the LOTUS SYSTEM® are designed for the massage practitioner to correspond audible, note/tonal frequency and resonance, changes in timing, notes and tones, scale progression, breakdowns, sustained notes, chords and sporadic sounds produced when a music selection is played with massage techniques, stroke values, pressures, directions and speed. In the embodiment shown music body charts are designed to correspond to treble and bass clefs typically used only to create audible music. Here, each “high notes” section 32 on a music body chart corresponds to a treble clef 26 and each “low notes” section 34 corresponds to a bass clef 28. A “mid notes” section 36 on a music body chart can correspond to transitions from treble and bass clefs and ½, ¼ and ⅛ notes, sustained notes, as well as major and minor scaled notes.

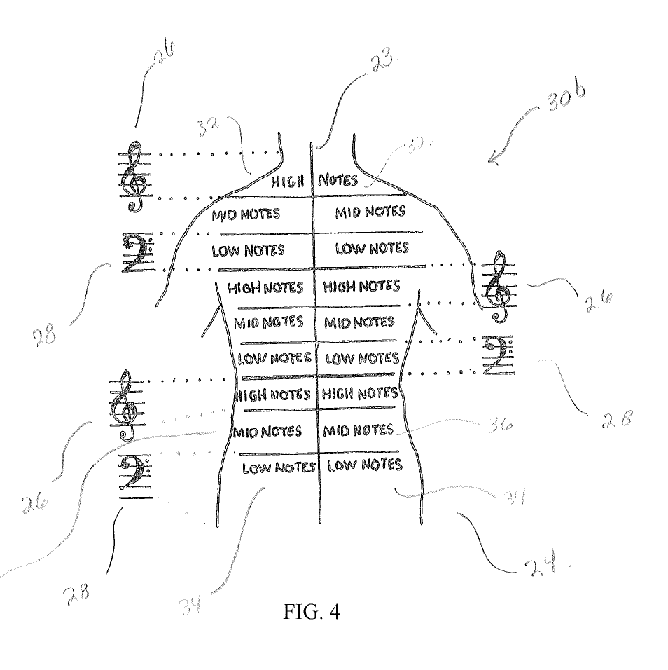

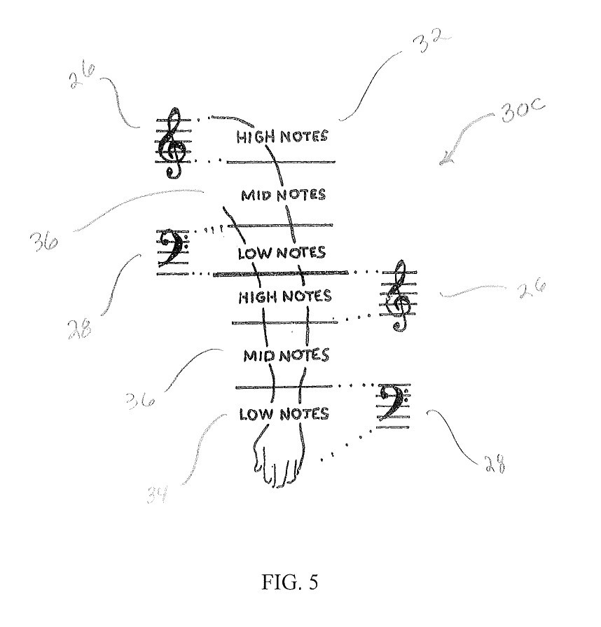

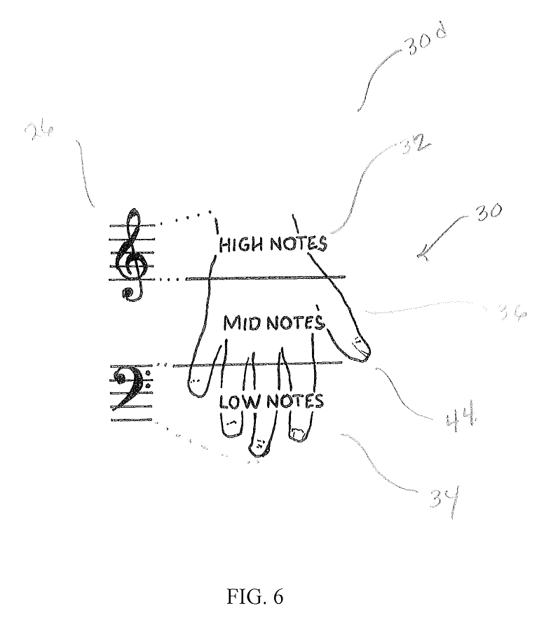

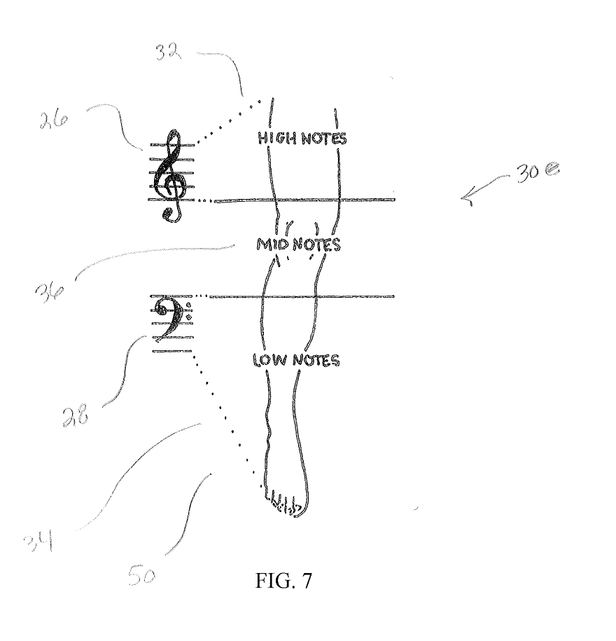

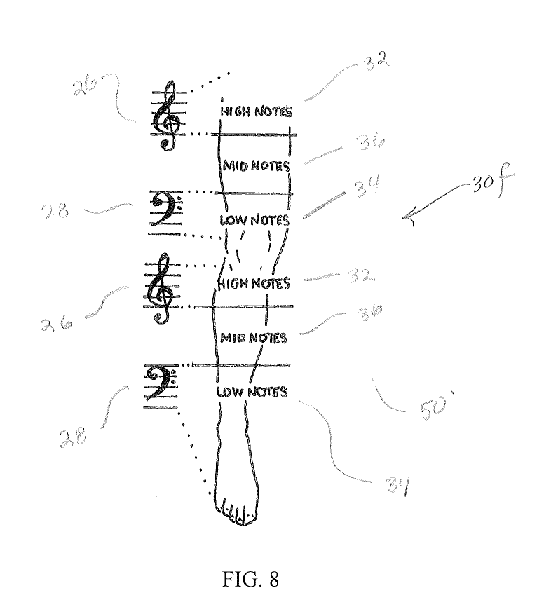

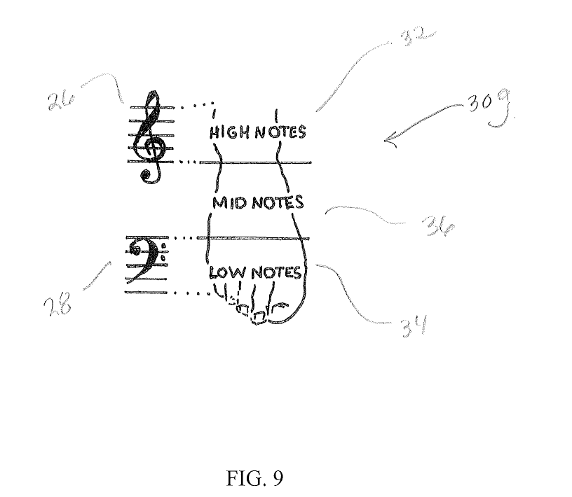

Examples of relationship of clefs, notes, and body surfaces for applied pressure are represented in FIGS. 3-9, using dotted lines 38. FIG. 3 shows a music body chart for the upper torso 24, having one high notes section 32, one low notes section 34, and one mid notes section 36. In this 3-section chart 30a, the treble clef 26 corresponds to the high note section 32 and the bass clef 28 corresponds to the low notes section 34. FIG. 4 shows another music body chart 30b for the upper torso 24, having notes sections separated by the mid-sagittal plane 23. In this version of the chart, three treble clefs 26 corresponds to six high note sections 32 and three bass clefs 28 corresponds to six low notes sections 34. FIG. 5 shows a music body chart 30c for an upper extremity 42 such as an arm. In this version of the chart, two treble clefs 26 corresponds to a respective high note section 32 and two bass clefs 28 correspond to a respective low notes section 34. FIG. 6 shows a music body chart 30d for a hand 44. This chart version 30d includes a treble clef 26 which corresponds to one high note section 32 in the wrist area 46 and one bass clef 28 which corresponds to a low notes sections 34 over four digits 48. FIG. 7 shows a music body chart 30e for a lower extremity such as a leg 50. This chart version 30e includes a treble clef 26 which corresponds to one high note section 32 in the upper thigh area and one bass clef 28 which corresponds to a low notes sections 34 over a section covering the foot, ankle, and a portion of the calf. FIG. 8 shows another music body chart 30f for a lower extremity such as a leg 50. This chart version 30f includes two treble clefs 26 and two bass clefs which correspond to designated portions along the leg. FIG. 9 shows a music body chart 30g for a lower extremity such as a foot 56. This chart version 30g includes one treble clef 26 which corresponds to one high note section 32 in an ankle area and one bass clef 28 which corresponds to a low notes sections 34 over a section covering the toes and base of the foot. Each of the chart versions shown in FIG. 3-9 also includes mid notes sections 36 covering area positioned between the high and low notes sections.

In using Lotus massage systems and methods, especially in combination with referencing a music body chart, a massage practitioner can engage in a sort of musical dance when applying pressure to a massage recipient. For example, these systems and methods can blend all major and minor notes/scales of audibly produced music/sounds played during the massage session above middle C and apply strokes and movements according to “high notes” sections of a music body chart. A massage practitioner should understand beats per minute (“BPM”), rhythm cycles and tempo changes in order to perform Lotus techniques at optimum levels during massage.

In so doing, the practitioner will use a pressure implementing device 60 (FIGS. 1a, 1b) such as their hands, forearms, elbows or any external device such as stones and massage sticks (e.g. bamboo), which may be used to apply pressure. Each type pressure applied to the massage recipient, whether a stroke or another type of movement can correspond to a music body chart. A practitioner is, therefore, able to map, synchronize, choreograph, and/or in a freeform manner apply pressure to a massage recipient, to match music body charts developed for Lotus massage systems and methods. Music body charts may, therefore, be used a transference and/or template enabling tool for the massage practitioner to apply various types of strokes and movement to a massage recipient. Notes, rhythms, tempos, etc. of a musical selection also correspond to music body chart such that the selection's rhythm and tempo, for example, can address the massage client's needs and/or preferences. Variances in pressures applied during the massage can be determined by reference to the following music body chart:

TABLE 1

| Stroke/Movement | Note Level | Area of Body | Pressure Variance |
|---|---|---|---|
| Petrissage | Low (Bass Clef below mid C) | Most Distal and Lumbar | Deep |
| | Mid (Transitions/other notes) | Mid Back/Mid Extremity | Med to Deep |
| | High (Treble clef above mid C) | Most Proximal/Cervical | Med/Superficial/Nerve Stroke |
| Effleurage | Low (Bass Clef below mid C) | Most Distal and Lumbar | Deep |
| | Mid (Transitions/other notes) | Mid Back/Mid Extremity | Med to Deep |
| | High (Treble clef above mid C) | Most Proximal/Cervical | Med/Superficial/Nerve Stroke |
| Light Effleurage/Nerve Stroke | Low (Bass Clef below mid C) | Most Distal and Lumbar | n/a |
| (Stimulating Stroke) | Mid (Transitions/other notes) | Mid Back/Mid Extremity | Superficial |
| | High (Treble clef above mid C) | Most Proximal/Cervical | Superficial |
| Compression | Low (Bass Clef below mid C) | Most Distal and Lumbar | Deep |
| Sustained Pressure | Mid (Transitions/other notes) | Mid Back/Mid Extremity | Med to Deep |
| Ischemic Pressure | High (Treble clef above mid C) | Most Proximal/Cervical | Med/Superficial/Nerve Stroke |
| (Trigger Point) | | | |
| Friction | Low (Bass Clef below mid C) | Most Distal and Lumbar | Deep |
| Vibration | Mid (Transitions/other notes) | Mid Back/Mid Extremity | Med to Deep |
| | High (Treble clef above mid C) | Most Proximal/Cervical | Med/Superficial/Nerve Stroke |
| Tapotement | Low (Bass Clef below mid C) | Most Distal and Lumbar | Deep |
| | Mid (Transitions/other notes) | Mid Back/Mid Extremity | Med to Deep |
| | High (Treble clef above mid C) | Most Proximal/Cervical | Med/Superficial/Nerve Stroke |
| Acupressure | Low (Bass Clef below mid C) | Most Distal and Lumbar | Deep |
| | Mid (Transitions/other notes) | Mid Back/Mid Extremity | Med to Deep |
| | High (Treble clef above mid C) | Most Proximal/Cervical | Med/Superficial/Nerve Stroke |

When referencing the music body charts shown in FIGS. 3-9 and Table 1, however, a massage practitioner should be careful not over exert themselves during a massage session by trying to apply pressure during every note and beat. Preferably, practitioners should choose specific parts of the musical selection to correspond to strokes and movements similar to those shown in FIG. 3-9 and Table 1.

Separating musical selections into low, mid, and high notes corresponding to massage recipient's lumbar, thoracic, and cervical regions, the recipient essentially becomes an open canvas to create mapped, choreographed, synchronized, and/or freeform strokes and/or movement, and stance sequences. As shown particularly in Table 1, various types of applications can be applied to various body surfaces depending on how a musical selection moves along a musical scale. As a massage practitioner listens and continues to separate notes in a musical selection application of mapped, choreographed, synchronized, and/or freeform strokes and/or movements can become easier. Moreover, the practitioner's ability to adjust pressure with the intensity of the notes in the musical selection will develop over time.

Variances in pressure may be determined as stroke or movement progresses. For example, when a tonal level changes, the massage practitioner's pressure will change. This direct application is evident when the note/tonal changes occur during the musical selection played during the massage session. For example, if when beginning a massage stroke, a note/tonal level drops from an “A” to a “D” minor in 4 counts, the massage stroke can start at the proximal end of an extremity or in the cervical region of the back. As the stroke progresses with the tonal change, the pressure can change from a superficial application of pressure, to a medium application, to a deep application as the stroke ends. The deeper pressure will be sustained as long as the low note is held. It is therefore important for practitioners to know musical sounds and corresponding pressures to determine the most suited stroke or movement to apply. This rational also applies to pressure application during low to high notes. As a tone begins to rise, the massage practitioner will change the direction and pressure of a stroke or movement to flow with the musical selection.

Sustained notes, such as those used in New Age genres can result in stroke and movement applied from cervical to lumbar and/or proximal to distal of an extremity. However, pressure can be applied during sustained notes from across the recipient's back or another extremity, perpendicular to the rhythm and tempo pathways and muscle fibers (e.g. cervical to lumbar, proximal to distal and vice versa).

When notes or tones rise, a massage practitioner can also apply strokes or movements towards the heart. When notes or tones lower, the practitioner can then apply strokes or movements away from the heart while keeping the variance of pressure with the intensity of the note. This type of pressure application can pertains to the extremities, i.e. arms, legs, hands, feet, the back, and “sub-sections” of those areas

As previously referenced, different speeds and tempo patterns of a musical selection can affect the massage recipients cerebral and Central Nervous Systems (“CNS”) reactions and responses. Different genres can, therefore, affect the massage practitioners overall speed throughout a massage session. This includes all movements, strokes and stances made by the massage practitioner during session. If massage client wishes to “wake up” and to assist in a feeling of alertness during or after the massage, faster rhythm and tempo patterns of a musical selection may be more suited to enhance the recipient's experience. Similarly, if a more relaxing and de-stressing type of massage is requested, a musical selection having slower rhythms and tempo patterns would be more suitable.

In preferred embodiments of the LOTUS SYSTEM®, additional system components may be implemented to further enhance the massage experience for both the massage practitioner and recipient. These components include: use of playlists contained on a non-transitory storage medium, 360° sight cancellation, ambient noise reduction, and aromatherapy.

In one aspect, the system may include musical selections contained on storage media such as a cellular device, tablet device (e.g. iPod, iPad), a compact disc, a computer, a phone, a tape cassette, or any other type of device suitable for music playback. The storage media may further include pre-determined playlists of music and/or sounds for relaxation and other purposes during massage. The playlists are preferably set at a specific time length to match the amount of time of the massage session. For example, multiple musical selections may be included in a playlist that last for a 60-minute, 90-minute, or 120-minute massage session. Using the playlist disposed on the storage medium, therefore, enables the massage practitioner to concentrate more on applying pressure according to Lotus massage systems and methods without referring to or concentrating on a visible time device. Use of playlists in this manner, would be similar to a dancer's choreographed movements which are synchronized to a musical selection.

Another component of Lotus massage systems and methods includes the use of 360° sight cancellation. In using this component, a covering is placed over the eyes of the client to remove ambient sources of light. Removal of light sources in this manner has been shown to further relax the massage recipient and slow the recipient's motor cortex and brain synapse output such that the recipient body is triggered into a state of homeostasis.

Similarly, ambient noise reduction through the use of noise canceling devices can also trigger a state of homeostasis. The types of noise canceling devices preferable remove the majority of ambient noise and noise immediate surroundings for both the massage practitioner and the massage recipient during the massage session. Examples of ambient noise includes, but is not limited to noise from creaking floors, movements in areas outside of the massage room, conversations being held outside the massage room, loud ventilation systems, slamming doors, toilet flushing, ringing phones, airplanes, automobiles, motorcycles, lawnmowers, cell phones, etc. The list of potential source of noise is potentially endless. Use of noise reduction devices, such as noise canceling headphones and earbuds enhances the overall massage experience, by allowing the recipient to relax, feel comfortable and disengage from their immediate environment. The massage practitioner may also wear noise reduction devices during the massage sessions. This component of the system can also increase practitioner's immersion with the music selection and their application of choreographed, mapped, synchronized and/or freeform strokes and movements. Use of this component also frees the massage practitioner from potential distractions that may inhibit optimal application of Lotus massage systems and methods.

Aromatherapy may also be used as another component of Lotus massage systems and methods. The types of oils and blends described below, however, provide only a template for potential benefits, which may be experienced by a massage recipient:
inner peace and happiness: ylang ylang, sandalwood, geranium, rose, orange, lemon, frankincense, neroli, bergamot, grapefruit
self-confidence: grapefruit, orange, rosemary, jasmine, bergamot, cypress, bay laurel
help against insecurity issues: frankincense, sandalwood, vetiver, bergamot, cedarwood, jasmine
help for battling fatigue, burnout, and exhaustion: sandalwood, rosemary, basil, jasmine, black pepper, clary sage, lemon, peppermint, vetiver, bergamot, ginger
help against anger issues: ylang ylang, neroli, rose, jasmine, orange, bergamot, vetiver
help for battling stress: rose, jasmine, neroli, bergamot, vetiver, ylang ylang, clary sage, frankincense, grapefruit, lavender

While embodiments of Lotus massage system and methods have been shown and described, it will be apparent to those skilled in the art that many more modifications are possible without departing from the inventive concepts herein. The invention, therefore, is not to be restricted, except in the spirit of the following claims.

What is claimed is:

1. A Lotus Massage System, comprising:
a storage medium configured to play at least one musical selection; and
a pressure implementing element configured to move against a body surface, the pressure implementing element being configured to apply pressure to the body surface in combination with the musical selection.

2. The System of claim 1, further comprising an auditory system configured to couple with auditory receptors of both a user and a massage therapist.

3. The System of claim 1, wherein the auditory system comprises at least two pairs of auditory devices.

4. The System of claim 1, wherein the auditory system comprises wireless auditory devices.

5. The System of claim 1, wherein the musical selection is configured to play on a cellular device.

6. The System of claim 1, wherein the pressure implementing element is a handheld object.

7. The System of claim 1, further comprising a sight cancellation device.

8. The System of claim 1, further comprising a music body chart.

9. The System of claim 8, wherein the applied pressure is choreographed according to the music body chart and the at least one musical selection.

10. The System of claim 8, wherein the applied pressure is mapped according to the music body chart and the at least one musical selection.

11. The System of claim 8, wherein the applied pressure comprises freeform application of strokes according to the music body chart and the at least one musical selection.

12. The System of claim 1, wherein the applied pressure is choreographed according to a music body chart and the at least one musical selection.

13. The System of claim 1, wherein the applied pressure is mapped according to a music body chart and the at least one musical selection.

14. The System of claim 1, wherein the applied pressure comprises freeform application of strokes according to the music body chart and the at least one musical selection.

15. A Lotus Massage Method, comprising:
playing at least one musical selection disposed on a storage medium; and
applying a pressure implementing element against a body surface, the pressure implementing element being configured to apply pressure to the body surface in combination with the at least one musical selection.

16. The Method of claim 15, further comprising providing an auditory system configured to couple with auditory receptors of both a user and a massage therapist.

17. The Method of claim 15, further comprising mapping the applied pressure according to a music body chart and the at least one musical selection.

18. The Method of claim 15, further comprising choreographing the applied pressure according to a music body chart and the at least one musical selection.

19. The Method of claim 15, further comprising sequencing the applied pressure in a freeform manner according to a music body chart and the at least one musical selection.

20. A Lotus Massage kit, comprising:
a storage medium having at least one musical selection;
a plurality of auditory devices configured to output the at least one musical selection;
a sight cancellation device; and
an aromatherapy device configured to distribute aroma.
